(19) United States
(12) Patent Application Publication
(10) Pub. No.: US 2025/0281687 A1
(43) Pub. Date: Sep. 11, 2025

(54) SUPPORT DEVICE FOR CATHETER OR INJECTION LINE

(71) Applicant: Boston Scientific Scimed, Inc., Maple Grove, MN (US)

(72) Inventors: David Raab, Roseville, MN (US); Logan Ernster, Saint Paul, MN (US); Charles Weber, St. Paul, MN (US)

(21) Appl. No.: 19/071,144

(22) Filed: Mar. 5, 2025

Related U.S. Application Data
(60) Provisional application No. 63/562,949, filed on Mar. 8, 2024.

(51) Int. Cl. A61M 5/14

(52) U.S. Cl. CPC A61M 5/1415; A61M 2202/0007; A61M 2209/082

(57) ABSTRACT

Embodiments herein relate to a support device for a cancer therapy catheter or injection line. In an embodiment, a medical device support stand is included having a vertical frame member and an adjustable height support arm, wherein the adjustable height support arm is configured to be removably attached to the vertical frame member at varying heights thereon. The support stand also includes a support leg, wherein the support leg is configured to be removably attached to a bottom portion of the vertical frame member. The adjustable height support arm can be configured to engage with a body member of a medical device and retain the same in a vertical orientation. Other embodiments are also included herein.

This application claims the benefit of U.S. Provisional Application No. 63/562,949, filed Mar. 8, 2024, the content of which is herein incorporated by reference in its entirety.

### FIELD

Embodiments herein relate to a support device for a cancer therapy catheter or injection line.

### BACKGROUND

According to the American Cancer Society, cancer accounts for nearly 25% of the deaths that occur in the United States each year. Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division. Surgery is a common first-line therapy for many cancerous tumors. However, not every tumor can be surgically removed.

In one approach to treat unresectable tumors, targeted radioembolization therapy can be used. For example, Y-90 glass microspheres can be delivered into a tumor through a microcatheter placed into an artery that supplies blood to the tumor. The Y-90 microspheres become trapped in the tumor and the beta radiation emitted by the Y-90 exerts a local radiotherapeutic effect to the tumor.

### SUMMARY

Embodiments herein relate to a support device for a cancer therapy catheter or injection line. In a first aspect, a medical device support stand can be included having a vertical frame member, an adjustable height support arm, wherein the adjustable height support arm can be configured to be removably attached to the vertical frame member at varying heights thereon, and a support leg, wherein the support leg can be configured to be removably attached to a bottom portion of the vertical frame member. The adjustable height support arm can be configured to engage with a portion of a cancer therapy delivery system to retain at least a portion of the cancer therapy delivery system in a vertical orientation.

In a second aspect, in addition to one or more of the preceding or following aspects, or in the alternative to some aspects, the portion the cancer therapy delivery system secured in a vertical orientation includes at least one of a hub, a manifold, or a connector.

In a third aspect, in addition to one or more of the preceding or following aspects, or in the alternative to some aspects, can further include a stability wing, wherein the stability wing can be configured to engage with the support leg and rotate between a first position wherein a lengthwise axis of the stability wing can be parallel to a lengthwise axis of the support leg and a second position wherein the lengthwise axis of the stability wing can be perpendicular to the lengthwise axis of the support leg.

In a fourth aspect, in addition to one or more of the preceding or following aspects, or in the alternative to some aspects, the adjustable height support arm can include a retention member, wherein the retention member can be configured to engage with a portion of the cancer therapy delivery system to retain at least a portion of the cancer therapy delivery system in a vertical orientation.

In a fifth aspect, in addition to one or more of the preceding or following aspects, or in the alternative to some aspects, the adjustable height support arm can include attachment jaws, wherein the attachment jaws can be configured to engage with a portion of the cancer therapy delivery system to retain at least a portion of the cancer therapy delivery system in a vertical orientation.

In a sixth aspect, in addition to one or more of the preceding or following aspects, or in the alternative to some aspects, the support stand can further include an angled connector.

In a seventh aspect, in addition to one or more of the preceding or following aspects, or in the alternative to some aspects, the angled connector can be configured to engage both the vertical frame member and the support leg thereby connecting the two together.

In an eighth aspect, in addition to one or more of the preceding or following aspects, or in the alternative to some aspects, the angled connector can include a first snap-fit mechanism, wherein the first snap-fit mechanism can be configured to engage with the vertical frame member, and a second snap-fit mechanism, wherein the second snap-fit mechanism can be configured to engage with the support leg.

In a ninth aspect, in addition to one or more of the preceding or following aspects, or in the alternative to some aspects, the adjustable height support arm can include a channel, wherein the vertical frame member can be configured to fit at least partially within the channel.

In a tenth aspect, in addition to one or more of the preceding or following aspects, or in the alternative to some aspects, the vertical frame member can include a plurality of apertures, wherein the plurality of apertures can be distributed vertically along a lengthwise axis of the vertical frame member.

In an eleventh aspect, in addition to one or more of the preceding or following aspects, or in the alternative to some aspects, the adjustable height support arm can include a detent mechanism, wherein the detent mechanism can be configured to engage with at least one of the plurality of apertures to hold the adjustable height support arm at a specific point along the lengthwise axis of the vertical frame member.

In a twelfth aspect, in addition to one or more of the preceding or following aspects, or in the alternative to some aspects, the detent mechanism can include a spring-force detent mechanism.

In a thirteenth aspect, in addition to one or more of the preceding or following aspects, or in the alternative to some aspects, the adjustable height support arm can include a first base piece, and a second telescoping piece, wherein the second telescoping piece can be moved in or out relative to the first base piece.

In a fourteenth aspect, a cancer-therapy delivery system can be included having a fluid reservoir, a catheter, and a support stand. The support stand can include a vertical frame member, an adjustable height support arm, wherein the adjustable height support arm can be configured to be removably attached to the vertical frame member at varying heights thereon, and a support leg, wherein the support leg can be configured to be removably attached to a bottom portion of the vertical frame member. The adjustable height support arm can be configured to engage with the catheter and retain at least a portion of the same in a vertical orientation.

In a fifteenth aspect, in addition to one or more of the preceding or following aspects, or in the alternative to some aspects, the support stand can further include a stability wing, wherein the stability wing can be configured to engage with the support leg and rotate between a first position wherein a lengthwise axis of the stability wing can be parallel to a lengthwise axis of the support leg and a second position wherein the lengthwise axis of the stability wing can be perpendicular to the lengthwise axis of the support leg.

In a sixteenth aspect, in addition to one or more of the preceding or following aspects, or in the alternative to some aspects, the adjustable height support arm can include a retention member, wherein the retention member can be configured to engage with a body member of a medical device and retain the same in a vertical orientation.

In a seventeenth aspect, in addition to one or more of the preceding or following aspects, or in the alternative to some aspects, the adjustable height support arm can include attachment jaws, wherein the attachment jaws can be configured to engage with a body member of a medical device and retain the same in a vertical orientation.

In an eighteenth aspect, in addition to one or more of the preceding or following aspects, or in the alternative to some aspects, the support stand can further include an angled connector, wherein the angled connector can be configured to engage both the vertical frame member and the support leg thereby connecting the two together.

In a nineteenth aspect, in addition to one or more of the preceding or following aspects, or in the alternative to some aspects, the adjustable height support arm can include a channel, wherein the vertical frame member can be configured to fit at least partially within the channel.

In a twentieth aspect, a method of administering a cancer therapy can be included. The method can include preparing a cancer-therapy delivery system by performing one or more of priming the system, removing bubbles, and flushing the lines, assembling a support stand by performing one or more of attaching the adjustable height support arm to the vertical frame member, attaching the support leg to the vertical frame member, and moving a stability wing into a deployed position, adjusting a height of the support stand, securing a catheter or injection line of the system to the support stand, and causing a fluid to flow through the system to a radioactive microsphere supply reservoir of the system and then out of the same carrying radioactive microspheres through a catheter secured by the support stand and into a desired site of a patient.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic diagram of a cancer-therapy delivery system in accordance with various embodiments herein.

FIG. 2 is an exploded view of a medical device support stand in accordance with various embodiments herein.

FIG. 3 is a perspective view of a medical device support stand in accordance with various embodiments herein.

FIG. 4 is a perspective view of a medical device support stand in accordance with various embodiments herein.

FIG. 5 is a perspective view of a medical device support stand in accordance with various embodiments herein.

FIG. 6 is a plan view of a portion of an adjustable height support arm in accordance with various embodiments herein.

FIG. 7 is a plan view of a portion of an adjustable height support arm in accordance with various embodiments herein.

FIG. 8 is a perspective view of a portion of an adjustable height support arm in accordance with various embodiments herein.

FIG. 9 is a top plan view of a portion of an adjustable height support arm in accordance with various embodiments herein.

FIG. 10 is a perspective view of a medical device support stand in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Targeted radioembolization therapy can be used on unresectable tumors. In one approach, Y-90 glass microspheres are delivered into a tumor through a microcatheter placed into an artery that supplies blood to the tumor. In some cases, it can be advantageous to hold the microcatheter in a fixed position with portions of the microcatheter, such as a hub or a manifold portion thereof, oriented vertically. The microspheres can be relatively heavy with respect the fluid in which they are delivered (such as a saline solution). As a result, they may fall out of suspension relatively easily under the influence of gravity. Internal portions of the fluid path through which the microspheres travel in therapy delivery systems herein can sometimes incidentally trap some of the microspheres, particularly when there is an internal surface feature such as a small gap or space that might occur such as at a connecter between two different components, at the entrance to a hub or manifold, or the like. However, it is believed that microspheres herein are less likely to be caught by and/or accumulate at such internal surface features if the direction of flow is vertical at such points as gravity will then not act to push the microspheres against the internal surface.

Embodiments herein can facilitate holding portions of a catheter or similar device, such as a hub, manifold, or connector portion thereof, in a vertical orientation. For example, in various embodiments herein a cancer-therapy delivery system includes a fluid reservoir, a catheter, and a support stand. The support stand can include a vertical frame member and an adjustable height support arm, wherein the adjustable height support arm is configured to be removably attached to the vertical frame member at varying heights thereon. The support stand can further include a support leg that can be configured to be removably attached to a bottom portion of the vertical frame member. The adjustable height support arm can be configured to engage with a catheter and can retain the same in a vertical orientation.

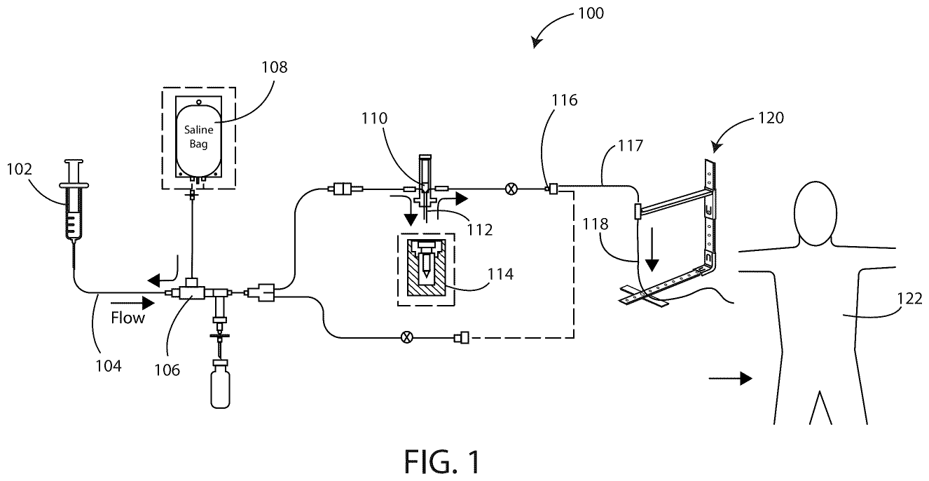

Referring now to FIG. 1, a schematic diagram of components of an exemplary cancer-therapy delivery system 100 is shown in accordance with various embodiments herein. Major parts of the cancer-therapy delivery system 100 include a therapeutic fluid delivery device 102, a fluid supply tube 104, and a flow control valve 106. In this example, the cancer-therapy delivery system 100 also includes a saline supply reservoir 108. The cancer-therapy delivery system 100 also includes a fluid injection and suspension withdrawal device 110. The cancer-therapy delivery system 100 also includes a radioactive microsphere supply reservoir 114. The cancer-therapy delivery system 100 also includes an outflow port 116. The cancer-therapy delivery system 100 also includes a microcatheter 118 and a support stand 120 to hold the microcatheter 118 in place. FIG. 1 also shows a patient 122 into which the microcatheter 118 can be inserted to deliver the therapeutic suspension of microspheres.

In use, various operations can be performed to prepare the system 100 for use. For example, operations can be formed such as system priming, bubble removal, flushing operations, and the like. In addition, the support stand 120 can be assembled as described herein, then the support stand 120 can be positioned, and the microcatheter 118 can be inserted into the support stand 120 and be secured therein. Then (omitting some possible operations for case of explanation) the clinician or other system user can pull back on a plunger or similar mechanism of therapeutic fluid delivery device 102 causing fluid (such as saline) to be withdrawn from the saline supply reservoir 108, through the flow control valve 106 and the fluid supply tube 104, and into the fluid delivery device 102. Then the clinician or other system user can depress the plunger causing fluid to flow from the therapeutic fluid delivery device 102, through the fluid supply tube 104, through the flow control valve 106, and into the fluid injection and suspension withdrawal device 110. The fluid injection and suspension withdrawal device 110 can be in fluid communication with the radioactive microsphere supply reservoir 114 and can direct a flow of fluid into the radioactive microsphere supply reservoir 114 coming from the therapeutic fluid delivery device 102 such as through one of a pair of needles, cannulas, or tubes 112. The fluid can become mixed with microspheres in the radioactive microsphere supply reservoir 114 forming a suspension which can then exit via the fluid injection and suspension withdrawal device 110 via another needle, cannula, or tube 112 and through tubing and out of the outflow port 116 and into the microcatheter 118 and into a desired site of the patient 122. In some embodiments, the support stand 120 can then be disassembled to facilitate easier disposal, such as by making it easier to insert the components of the support stand 120 into a radioactive waste disposal structure, container, bin, bag or the like.

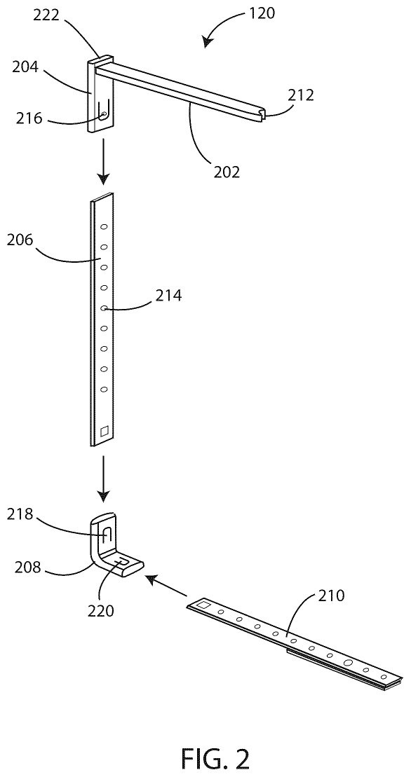

Referring now to FIG. 2, an exploded view of a medical device support stand 120 is shown in accordance with various embodiments herein. The medical device support stand 120 includes an adjustable height support arm 202. The medical device support stand 120 also includes a vertical frame member 206. In various embodiments, the adjustable height support arm 202 can be configured to be removably attached to a vertical frame member 206 at varying heights thereon. For example, the adjustable height support arm 202 can be attached at heights from 4 to 30 inches over a bottom of the medical device support stand 120. The receiver 204 can engage with the vertical frame member 206. In some embodiments, the adjustable height support arm 202 can include a receiver 204 that can define a channel 222 or slot. For example, the vertical frame member 206 can be configured to fit at least partially within the channel 222. In some embodiments, the vertical frame member 206 can be substantially rectangular in cross-section. However, various other shapes are also contemplated herein. During assembly of the medical device support stand 120 before use, the top (or the bottom) of the vertical frame member 206 can be inserted into the channel 222 and then the receiver 204 can slide down (or up) relative to the vertical frame member 206.

The receiver 204 can be removably attached to the vertical frame member 206 at various heights thereon to allow the catheter or injection line to be secured at various heights. This can be accomplished in various ways. In some embodiments, the receiver 204 can be removably attached using a fastener, such as a screw, clip, pin, bolt, or the like. However, to facilitate quick height adjustment, in some embodiments a hand operated detent mechanism can be used to engage the vertical frame member 206 and secure the same at a desired height. For example, the vertical frame member 206 can include a plurality of apertures 214 that can be engaged by a detent mechanism 216. In various embodiments, the plurality of apertures 214 are distributed vertically along a lengthwise axis of a vertical frame member 206. In various embodiments, the detent mechanism 216 can include a spring-force detent mechanism 216. The detent mechanism 216 can engage with the apertures 214 in order to secure the adjustable height support arm 202, and specifically the receiver 204 thereof, at a particular position along the vertical frame member 206. For example, in various embodiments, the detent mechanism 216 can be configured to engage with at least one of a plurality of apertures 214 when in a first position to hold the adjustable height support arm 202 at a specific point along the lengthwise axis of the vertical frame member 206 and, when in a second position, can be not in engagement with the aperture 214 to allow the adjustable height support arm 202 to move up or down relative to the vertical frame member 206.

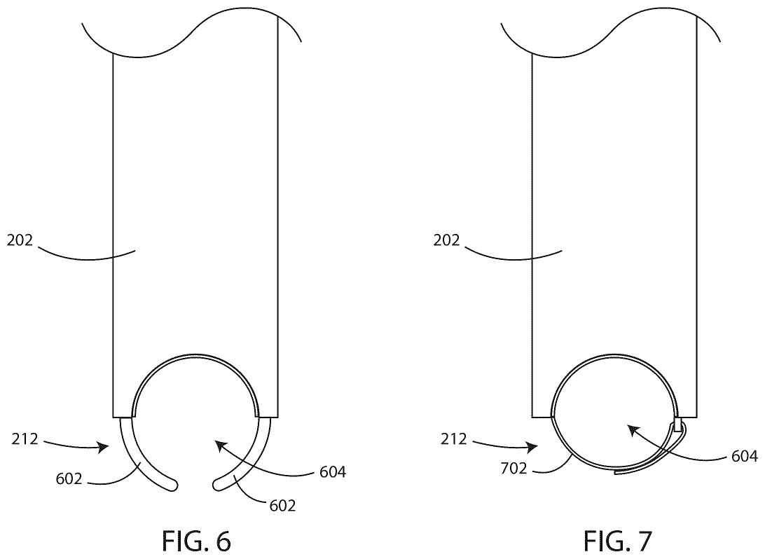

As referenced above, the adjustable height support arm 202 includes a retention member 212 to engage with a component of a cancer-therapy delivery system, such as a catheter, fluid line, or body member of the same and retain it in a vertical orientation. In some embodiments, the retention member 212 can specifically engage with a portion of a component of a delivery system to maintain a portion thereof, such as a hub, connector, or manifold of a cancer-therapy delivery system in a vertical position. To facilitate this, the retention member 212 can include features allowing it to receive and/or secure a component of the cancer-therapy delivery system, such as a catheter or other injection line. Further details of exemplary retention members are described below with reference to FIGS. 6-7.

The medical device support stand 120 also includes a support leg 210. In various embodiments, the support leg 210 can be configured to be removably attached to a bottom portion of a vertical frame member 206. In some embodiments, the medical device support stand 120 can also include an angled connector 208 to secure the support leg 210 to the vertical frame member 206. For example, the angled connector 208 can be configured to engage both a vertical frame member 206 and a support leg 210 thereby connecting the two together. It will be appreciated that various connection mechanisms can be used including snap-fit mechanisms, friction-fit mechanisms, fasteners such as screws, bolts, pins, or clips, and the like. However, in some embodiments, the angled connector 208 specifically includes a first snap-fit mechanism 218 and a second snap-fit mechanism 220. In various embodiments, the first snap-fit mechanism 218 can be configured to engage with a vertical frame member 206 and the second snap-fit mechanism 220 can be configured to engage with a support leg 210. Thus, during assembly before use, the vertical frame member 206 can be inserted into the first snap-fit mechanism 218 until it is locked in place while the support leg 210 can be inserted into the second snap-fit mechanism 218 until it is locked in place.

Components of the medical device support stand 120 can be formed of various materials. In some embodiments, components of the medical device support stand 120 can be formed of materials that can be sterilized. In various embodiments, the adjustable height support arm 202 can be formed of at least one of a metal (such as stainless steel, aluminum, titanium, or the like), a composite, or a polymer. Similarly, in various embodiments, the vertical frame member 206 can be formed of at least one of a metal (such as stainless steel, aluminum, titanium, or the like), a composite, or a polymer. Further, the support leg 210 can be formed of at least one of a metal (such as stainless steel, aluminum, titanium, or the like), a composite, or a polymer.

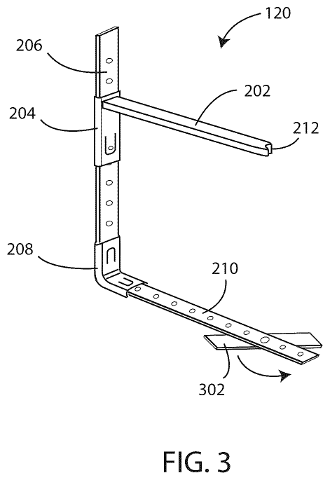

Referring now to FIG. 3, a perspective view of a medical device support stand 120 is shown in accordance with various embodiments herein. As before, the medical device support stand 120 includes an adjustable height support arm 202, a receiver 204, a vertical frame member 206, an angled connector 208, a support leg 210, and retention member 212. In this embodiment, the medical device support stand further includes a stability wing 302. The stability wing 302 can be configured to be attached to the support leg 210 and rotate between a first stored position, wherein a lengthwise axis of the stability wing can be parallel to a lengthwise axis of the support leg 210, and a second deployed position, wherein the lengthwise axis of the stability wing 302 can be perpendicular to the lengthwise axis of the support leg 210. In this manner, the stability wing 302 can provide stability to the support leg 210 and, therefore, provide stability to the entire medical device support stand 120.

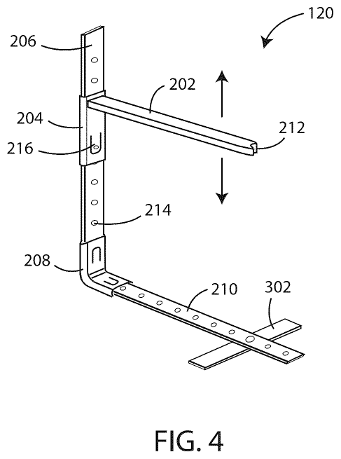

Referring now to FIG. 4, a perspective view of a medical device support stand 120 is shown in accordance with various embodiments herein. As before, the medical device support stand 120 includes an adjustable height support arm 202, a receiver 204, a vertical frame member 206, an angled connector 208, a support leg 210, retention member 212, and a stability wing 302. This view illustrates how adjustable height support arm 202 can be moved up and down relative to the vertical frame member 206 to allow a component of the cancer-therapy delivery system 100 to be held at a desired vertical height. For example, the spring-force detent mechanism 216 can be manipulated by the device user such that the receiver 204 can be slid up or down the vertical frame member 206. Then, when it is at a desired height, the detent mechanism 216 can be actuated to engage with apertures 214 to fix the receiver 204 in position.

In some embodiments, the medical device support stand can be placed on a flat surface. In some embodiments, the medical device support stand can be anchored in place. In some embodiments, a portion of the medical device support stand, such as the support leg can engage with a portion of a patient bed or a cart or table.

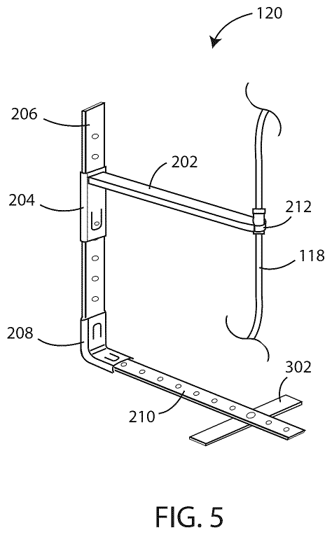

Referring now to FIG. 5, a perspective view of a medical device support stand 120 is shown in accordance with various embodiments herein. As before, the medical device support stand 120 includes an adjustable height support arm 202, a receiver 204, a vertical frame member 206, an angled connector 208, a support leg 210, retention member 212, and a stability wing 302. In this view, the retention member 212 has engaged with a catheter 118, such as a microcatheter herein for dosing. The retention member 212 can be configured to engage with various parts of the catheter 118. In various embodiments, the retention member 212 can engage with polymeric tubing of the catheter 118, a shaft of the catheter 118, a connector of the catheter 118, or the like. In some embodiments, one or more components of the support stand 120 herein can be physically integrated instead of existing as separate components. As such, in some embodiments, components herein may not be discrete pieces that are then assembled to create the support stand 120. However, it will be appreciated that it can be advantageous in some scenarios for the support stand 120 to be formed of discrete pieces that can be assembled and disassembled. For example, the components of the support stand 120 can be packed flat and shipped in a relatively flat container if they are provided in a disassembled form. As another example, disassembly of the support stand can facilitate easier disposal, such as by making it easier to insert the components of the support stand 120 into a radioactive waste disposal structure, container, bin, bag or the like.

Referring now to FIG. 6, a plan view of a portion of an adjustable height support arm 202 is shown in accordance with various embodiments herein. In this view, the retention member 212 includes attachment jaws 602. The adjustable height support arm 202 and/or retention member 212 attached thereto also includes a channel 604 into which a portion of the catheter 118 or other component of the cancer-therapy delivery system 100 can fit. In some embodiments, the channel 604 can be substantially U-shaped or semicircular. However, the channel 604 can also take on various other shapes. The attachment jaws 602 are configured to engage with the catheter 118 and retain the same in a vertical orientation. In some embodiments, the attachment jaws 602 can be flexible, such that the catheter 118 can be pushed into the attachment jaws 602 and the attachment jaws 602 can snap into place around the catheter 118. In some embodiments, the attachment jaws 602 can be pulled apart or otherwise opened to receive the catheter 118 before being closed around the catheter 118 to secure it in place.

It will be appreciated, however, that the retention member 212 can take various forms and configurations while functioning to hold the catheter. For example, in some embodiments, the retention member 212 can include a strap to secure the catheter in place. Referring now to FIG. 7, a plan view of a portion of an adjustable height support arm 202 is shown in accordance with various embodiments herein. As before, the adjustable height support arm 202 and/or retention member 212 attached thereto also includes a channel 604 into which a portion of the catheter or other component of the cancer-therapy delivery system can fit. After the catheter is positioned within the channel 604, a strap 702 can be used to secure the catheter in place. Beyond a strap or attachment jaws, however, it will be appreciated that various other mechanisms can be used to hold the catheter in place including, but not limited to, a clip, a band, a cover, an adhesive, or other mechanical mechanisms.

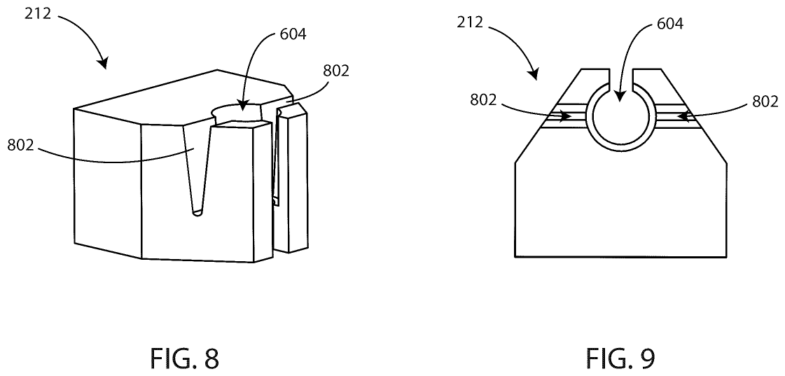

Referring now to FIG. 8, a perspective view is shown of a portion of an adjustable height support arm in accordance with various embodiments herein. As before, a portion of the adjustable height support arm and/or retention member 212 includes a channel 604 into which a portion of the catheter or other component of the cancer-therapy delivery system can fit. In this embodiment, the retention member 212 also includes slots 802 to accommodate wings or similar structures associated with the manifold or connector of a catheter. The slots 802 can include a solid bottom against which the wings or similar structure can rest after the catheter or other component is inserted into the retention member 212. The slots 802 can be tapered so they are narrower at the bottom, such as defining a V-shape allowing of easy insertion of the wings or similar structure from the top, yet holding them securely after insertion. FIG. 9 is a top plan view of a portion of the adjustable height support arm in accordance with various embodiments herein. FIG. 9 shows the retention member 212 from a top viewpoint. As before, the retention member 212 includes a channel 604 into which a portion of the catheter or other component of the cancer-therapy delivery system can fit along with slots 802 to accommodate wings or similar structures associated with the manifold or connector of a catheter.

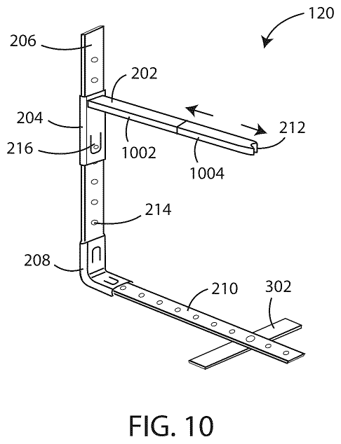

In some embodiments, the medical device support stand can support movement in additional directions to allow the catheter to be held in a specific position as selected by a device user. Referring now to FIG. 10, a perspective view of a medical device support stand 120 is shown in accordance with various embodiments herein. In this embodiment, medical device support stand 120 includes an adjustable height support arm 202, a receiver 204, a vertical frame member 206, an angled connector 208, a support leg 210, retention member 212, apertures 214, spring-force detent mechanism 216, and a stability wing 302 as before. However, in this view, the adjustable height support arm 202 includes a first base piece 1002 and a second telescoping piece 1004. The second telescoping piece 1004 can be moved in or out relative to the first base piece 1002 and then, when in a desired position, can be locked into place, such as by using a spring-loaded detent, spring-loaded nut, slip, pin, bolt, screw, or the like.

### Methods

Many different methods are contemplated herein, including, but not limited to, methods of making, methods of using, methods of administering a cancer therapy, methods of securing a cancer therapy catheter or injection line and the like. Aspects of system/device operation described elsewhere herein can be performed as operations of one or more methods in accordance with various embodiments herein.

In some embodiments, a method of administering a cancer therapy can include preparing the cancer-therapy delivery system (including, for example, one or more operations such as priming the system, removing bubbles, flushing the lines, and the like). In addition, the method can include assembling the support stand, such as by doing one or more of attaching the adjustable height support arm to the vertical frame member, attaching the support leg to the vertical frame member, and moving a stability wing into a deployed position. The method can also include positioning the support stand and/or adjusting a height of the support stand. The method can also include securing a catheter (such as a microcatheter) or injection line to the support stand, such as by engaging the catheter or injection line with a retention member. In some embodiments, the method can also include causing a fluid to flow through the system to a radioactive microsphere supply reservoir and then out of the same carrying radioactive microspheres to a catheter secured by the support stand and into a desired site of a patient.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A medical device support stand comprising:
a vertical frame member;
an adjustable height support arm, wherein the adjustable height support arm is configured to be removably attached to the vertical frame member at varying heights thereon; and
a support leg, wherein the support leg is configured to be removably attached to a bottom portion of the vertical frame member; and
wherein the adjustable height support arm is configured to engage with a portion of a cancer therapy delivery system to retain at least a portion of the cancer therapy delivery system in a vertical orientation.

2. The medical device support stand of claim 1, wherein the portion the cancer therapy delivery system comprises at least one of a hub, a manifold, or a connector.

3. The medical device support stand of claim 1, further comprising a stability wing, wherein the stability wing is configured to engage with the support leg and rotate between a first position wherein a lengthwise axis of the stability wing is parallel to a lengthwise axis of the support leg and a second position wherein the lengthwise axis of the stability wing is perpendicular to the lengthwise axis of the support leg.

4. The medical device support stand of claim 1, the adjustable height support arm comprising a retention member, wherein the retention member is configured to engage with a portion of the cancer therapy delivery system to retain at least a portion of the cancer therapy delivery system in a vertical orientation.

5. The medical device support stand of claim 1, the adjustable height support arm comprising attachment jaws, wherein the attachment jaws are configured to engage with a portion of the cancer therapy delivery system to retain at least a portion of the cancer therapy delivery system in a vertical orientation.

6. The medical device support stand of claim 1, further comprising an angled connector.

7. The medical device support stand of claim 6, wherein the angled connector is configured to engage both the vertical frame member and the support leg thereby connecting the two together.

8. The medical device support stand of claim 6, the angled connector comprising:
a first snap-fit mechanism, wherein the first snap-fit mechanism is configured to engage with the vertical frame member; and
a second snap-fit mechanism, wherein the second snap-fit mechanism is configured to engage with the support leg.

9. The medical device support stand of claim 1, the adjustable height support arm comprising a channel, wherein the vertical frame member is configured to fit at least partially within the channel.

10. The medical device support stand of claim 1, the vertical frame member comprising a plurality of apertures, wherein the plurality of apertures are distributed vertically along a lengthwise axis of the vertical frame member.

11. The medical device support stand of claim 10, the adjustable height support arm comprising a detent mechanism, wherein the detent mechanism is configured to engage with at least one of the plurality of apertures to hold the adjustable height support arm at a specific point along the lengthwise axis of the vertical frame member.

12. The medical device support stand of claim 11, the detent mechanism comprising a spring-force detent mechanism.

13. The medical device support stand of claim 1, the adjustable height support arm comprising:
a first base piece; and
a second telescoping piece, wherein the second telescoping piece can be moved in or out relative to the first base piece.

14. A cancer-therapy delivery system comprising:
a fluid reservoir;
a catheter; and
a support stand, the support stand comprising
a vertical frame member;
an adjustable height support arm, wherein the adjustable height support arm is configured to be removably attached to the vertical frame member at varying heights thereon; and
a support leg, wherein the support leg is configured to be removably attached to a bottom portion of the vertical frame member; and
wherein the adjustable height support arm is configured to engage with the catheter and retain at least a portion of the same in a vertical orientation.

15. The cancer-therapy delivery system of claim 14, the support stand further comprising a stability wing, wherein the stability wing is configured to engage with the support leg and rotate between a first position wherein a lengthwise axis of the stability wing is parallel to a lengthwise axis of the support leg and a second position wherein the lengthwise axis of the stability wing is perpendicular to the lengthwise axis of the support leg.

16. The cancer-therapy delivery system of claim 14, the adjustable height support arm comprising a retention member, wherein the retention member is configured to engage with a body member of a medical device and retain the same in a vertical orientation.

17. The cancer-therapy delivery system of claim 14, the adjustable height support arm comprising attachment jaws, wherein the attachment jaws are configured to engage with a body member of a medical device and retain the same in a vertical orientation.

18. The cancer-therapy delivery system of claim 14, the support stand further comprising an angled connector, wherein the angled connector is configured to engage both the vertical frame member and the support leg thereby connecting the two together.

19. The cancer-therapy delivery system of claim 14, the adjustable height support arm comprising a channel, wherein the vertical frame member is configured to fit at least partially within the channel.

20. A method of administering a cancer therapy comprising:
preparing a cancer-therapy delivery system by performing one or more of priming the system, removing bubbles, and flushing the lines;
assembling a support stand by performing one or more of attaching the adjustable height support arm to the vertical frame member, attaching the support leg to the vertical frame member, and moving a stability wing into a deployed position;
adjusting a height of the support stand;
securing a catheter or injection line of the system to the support stand; and
causing a fluid to flow through the system to a radioactive microsphere supply reservoir of the system and then out of the same carrying radioactive microspheres through a catheter secured by the support stand and into a desired site of a patient.
